(19) United States
(12) Patent Application Publication
(10) Pub. No.: US 2011/0218588 A1
(43) Pub. Date: Sep. 8, 2011

(54) IMPLANTABLE CORTICAL ELECTRICAL STIMULATION APPRATUS HAVING WIRELESS POWER SUPPLY CONTROL FUNCTION

(75) Inventors: Ho Chun Jung, Iksan-si (KR); Hyoung Ihl Kim, Seoul (KR)

(73) Assignee: YESU HOSPITAL MANAGEMENT FOUNDATION, Jeonju-si (KR)

(21) Appl. No.: 12/717,504

(22) Filed: Mar. 4, 2010

(51) Int. Cl. A61N 1/372; A61N 1/36

(52) U.S. Cl. 607/45

(57) ABSTRACT

An implantable cortical electrical stimulation device having wireless power control function comprises: an electrical stimulation device (100) which is arranged through a perforation part of cranial bone (1) and upper part of which is covered with scalp and thus electrical stimulation is transferred outside dura meter (2) to cerebral cortex (3); a head set (200) which is provided controllably with antenna (210) connecting wirelessly with the electrical stimulation device (100) outside scalp and applying power, and is fixed to a head; and a control element (300) which controls the electrical simulation device (310) through a power wire (211) connected to the antenna (210) of the head set (200). According to the present invention, cranial bone is perforated and electrodes of the electrical stimulation device allows reference electrode and stimulation electrode to be contacted with cerebral cortex, and the electrical stimulation device is fixed to cranial bone with titanium screw and then implantation thereof is finished. Here, in order to supply power to the electrical stimulation device an antenna for supplying power is approached closely to scalp and the antenna is placed to be an exact center of the electrical stimulation device so that power supply and data can be transferred without error.

### BACKGROUND OF THE INVENTION

The present invention relates to an implantable cortical electrical stimulation apparatus having a wireless power supply control function, and more particularly, to an implantable cortical electrical stimulation apparatus having a wireless power supply control function in which the cortical electrical stimulation apparatus is inserted into human body and power is transferred wirelessly, which is considered to be harmless to a human body, to drive the cortical electrical stimulation apparatus and further when power is transferred, data pursuant to the electrical stimulation conditions is received and based on the data intensity of the stimulation and setting thereof are controlled through external control device.

Generally, referring to cortical electrical stimulation, it has been used for activating or suppressing a connection between verves and thus attaining medical curing effects of melancholy, Parkinson's disease, function damage after stroking, epilepsy, or chronic neurological pain, etc.

Conventional cortical stimulation apparatus refers to transcramial stimulation devices such as transcramial magnetic stimulation or transcramial direct current stimulation. However, even the cortical stimulation through the transcramial stimulation devices are non-invasive, there arises a problem in that stimulation it transferred to unnecessary wide region of cortex over target-area of cerebral cortex.

Additionally, when transcramial stimulation device is used, there arises another problem in that more strong intensive stimulation has to be applied in order to apply desired intensive stimulation to cerebral cortex.

Meanwhile, when transcramial stimulation device is used, there arises another problem in that proper rehabilitation or exercise is difficult to be accompanied.

Accordingly, the present applicant has disclosed a solution to meet the aforementioned drawbacks that an electrical stimulation electrode is inserted into cranial cavity, which has been granted into Korean Patent No. 865,951 entitled “Self-expandable epidural electrode for cortical stimulation” (hereinafter, referred as “prior epidural electrode device”).

Here, the prior epidural electrode device is inserted to an upper part of dura meter surrounding cerebral cortex and transfers electrical stimulation to the cerebral cortex wherein it is formed as a lattice shape and further comprises a wire member transferring stimulation to cerebral cortex, a cover member housed expandably into the wire member, and power code one end of which is connected electrically to the wire member and an electrode is connected to an electrical stimulator and which supplies current to the wire member.

Here, the wire member is formed as a dual layer of an electrode layer receiving current from the electrical stimulator and transferring stimulation to cerebral cortex, and dielectric layer is formed over and insulating the electrode layer.

Accordingly, prior epidural electrode device provides controlled electrical stimulation to a local cerebral cortex in order to induce variation of brain nerve system so that deficiency of the nerve system function, which is caused from after brain is damaged from various reasons, can be treated.

However, according to the prior epidural electrode device configured in aforementioned way, current supplying member has to be inserted into a human body. Here, an insert type current supply device is inserted into a human body through operational method and further is restricted approaching to place or device under being influenced by magnetic field.

### SUMMARY OF THE INVENTION

Accordingly, in order to solve aforementioned drawbacks an object of the present invention relates to provide an implantable cortical electrical stimulation device having wireless power control in which when power is supplied wirelessly without battery, data based on electrical stimulation conditions is received and intensity and setting is controlled through external control device and thus cerebral cortex can be treated through electrical stimulation thereto.

In addition, coil for applying power is fixed confirmly to a head and thus treatment effect can not be decreased even head is vibrated.

In order to provide stimulation with wide area of cerebral cortex stimulation electrodes are spread in as state of reference electrode being fixed, and thus stimulation is transferred to wide area of cerebral cortex.

In order to achieve the object of the present invention, an implantable cortical electrical stimulation device having wireless power control function according to the present invention comprises: an electrical stimulation device which is arranged through a perforation part of cranial bone and upper part of which is covered with scalp and thus electrical stimulation is transferred outside dura meter to cerebral cortex; a head set which is provided controllably with antenna connecting wirelessly with the electrical stimulation device outside scalp and applying power, and is fixed to a head; and a control element which controls the electrical simulation device through a power wire connected to the antenna of the head set.

Additionally, an implantable cortical electrical stimulation device having wireless power control function according to another embodiment of the present invention comprises: a first electrical stimulation device which has one reference electrode and which is arranged through a perforation part of cranial bone and upper part of which is covered with scalp and thus electrical stimulation is transferred outside dura meter to cerebral cortex; a second and third electrical stimulation devices which are arranged throughly into both cranial bone as two parts to provide stimulation through wide area of cranial bone and upper part of which is covered with scalp and thus electrical stimulation is transferred outside dura meter to cerebral cortex; a head set which is provided controllably with antenna connecting electrically with the second and third electrical stimulation devices outside scalp and applying wirelessly power; and a control element which controls the second and third electrical simulation devices through a power wire connected to the antenna of the head set.

According to an implantable cortical electrical stimulation device having wireless power control function according to present invention, cranial bone is perforated and electrodes of the electrical stimulation device allows reference electrode and stimulation electrode to be contacted with cerebral cortex, and the electrical stimulation device is fixed to cranial bone with titanium screw and then implantation thereof is finished. Here, in order to supply power to the electrical stimulation device an antenna for supplying power is approached closely to scalp and the antenna is placed to be an exact center of the electrical stimulation device so that power supply and data can be transferred without error. Therefore, cerebral cortex is stimulated and activated to promote treatment effects and at the same time wide area of cerebral cortex is stimulated in a state of two electrical stimulation device being fixed to cranial bone, attaining various treatment effects.

### BRIEF DESCRIPTION OF THE DRAWING

FIG. 1 is a perspective view showing an overall configuration of implantable electrical stimulation device having wireless power supply control function according to the present invention.

FIG. 2 is a sectional view showing an electrical stimulation device according to the present invention, which is implanted into cerebral cortex.

FIG. 3 is a rear view showing a head set according to the present invention.

FIG. 4(a) is a perspective view showing an implantable electrical stimulation device according to the present invention.

FIG. 4(b) is a bottom view showing an implantable electrical stimulation device according to the present invention.

FIG. 5 is a sectional view showing an implantable electrical stimulation device according to the present invention.

FIG. 6 is a sectional view showing a reference electrode a stimulation electrode of an electrical stimulation device according to the present invention.

FIG. 7 is a sectional view showing a stimulation electrode of an electrical stimulation device according to the present invention.

FIG. 8 is a sectional view showing a reference electrode of an electrical stimulation device according to the present invention.

FIG. 9 is a view showing configurations of a wireless power supply control part and an implantable electrical stimulation device according to the present invention.

FIG. 10 is a sectional view showing an antenna provided only to a reference electrode of an implantable electrical stimulation device according to another embodiment of the present invention.

FIG. 11 is a sectional view showing a reference electrode of an implantable electrical stimulation device according to the present invention.

FIG. 12 is a sectional view showing a stimulation electrode of an implantable electrical stimulation device according to another embodiment of the present invention.

### DESCRIPTION OF THE PREFERRED EMBODIMENT

Hereinafter, preferred embodiments of a vacuum cyclone dust collector according to the present invention will be described referring to attached drawings. Here,

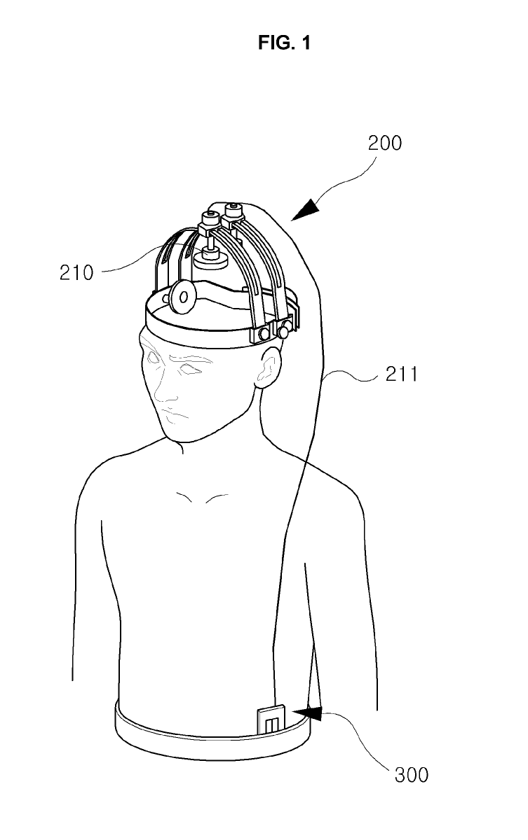

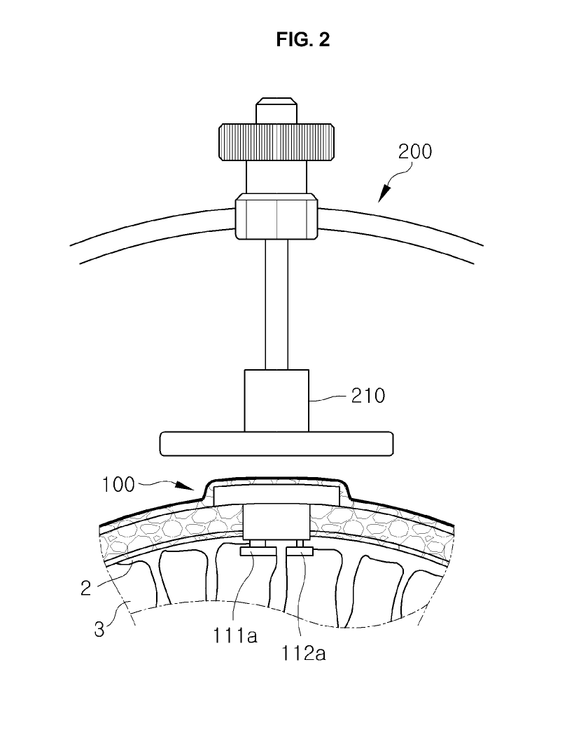

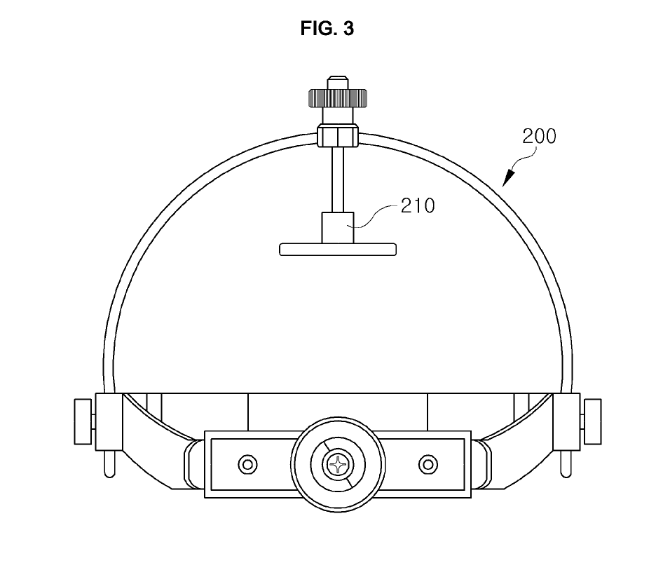

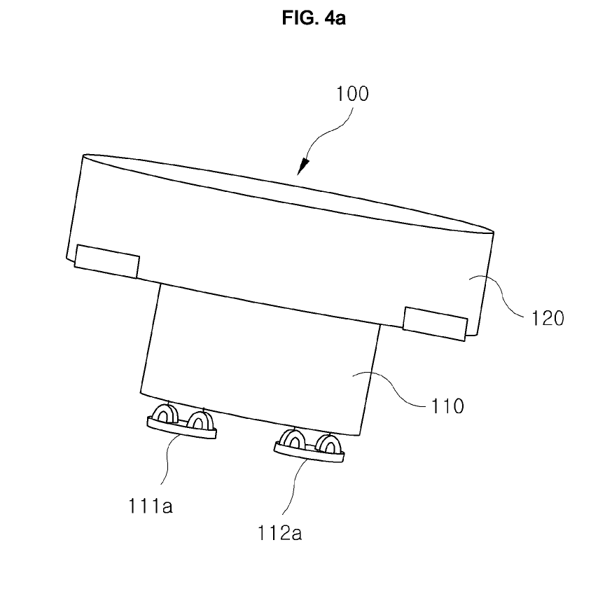

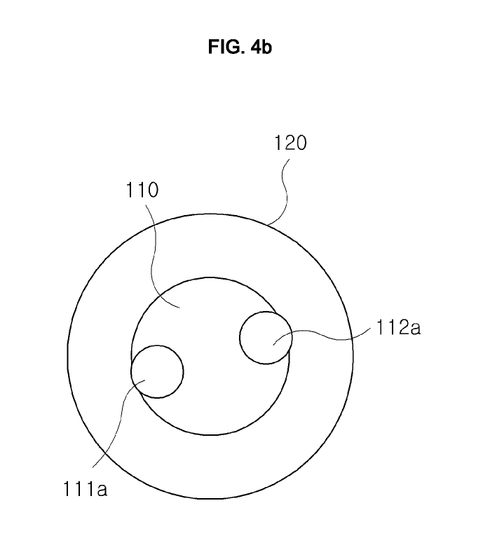

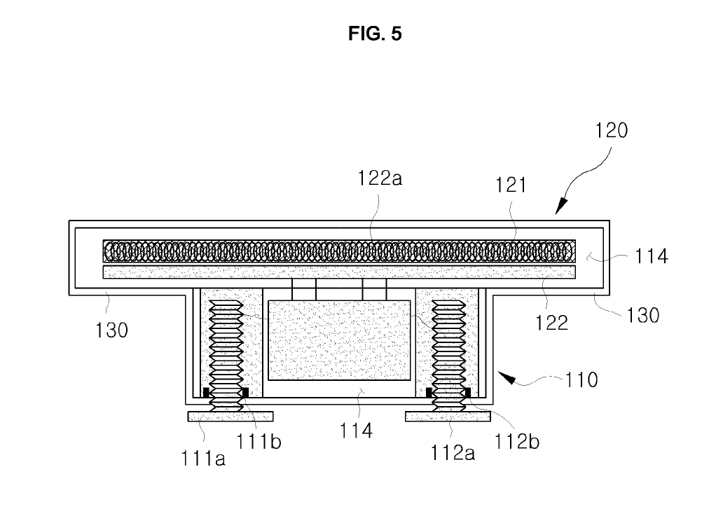

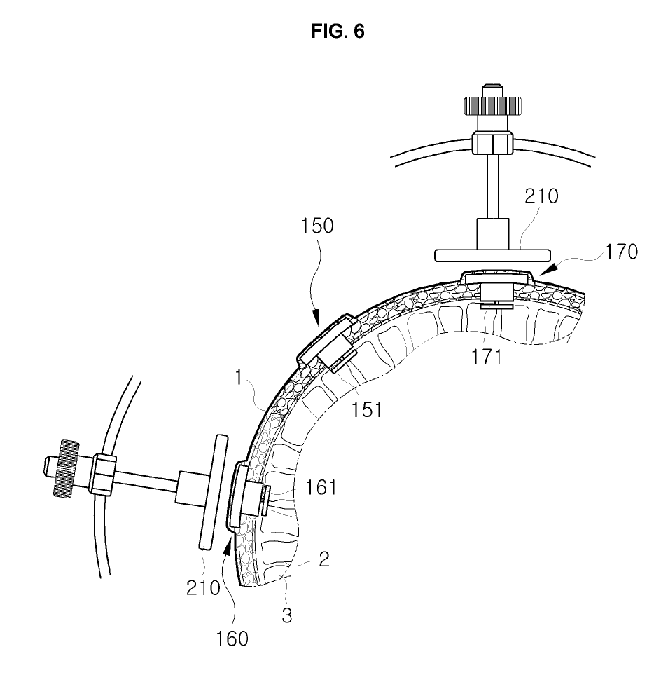

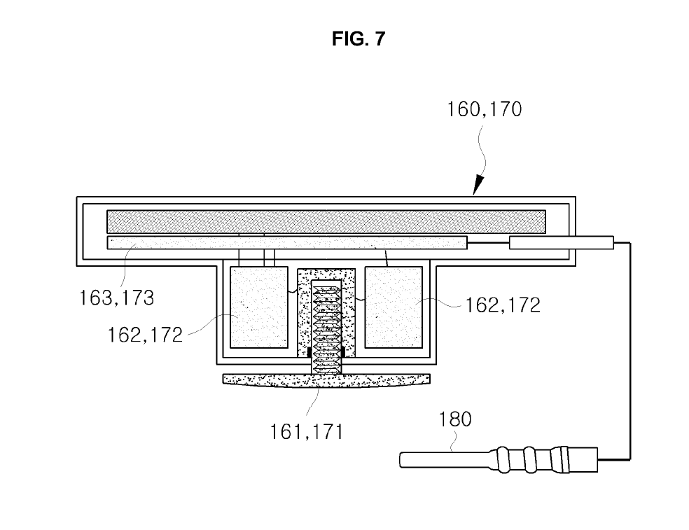

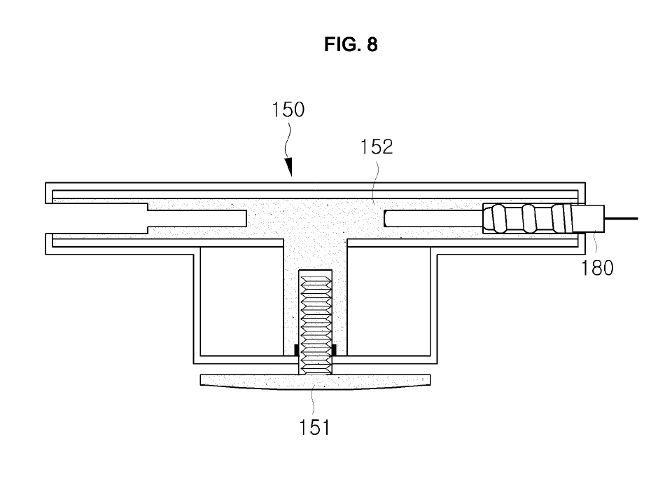

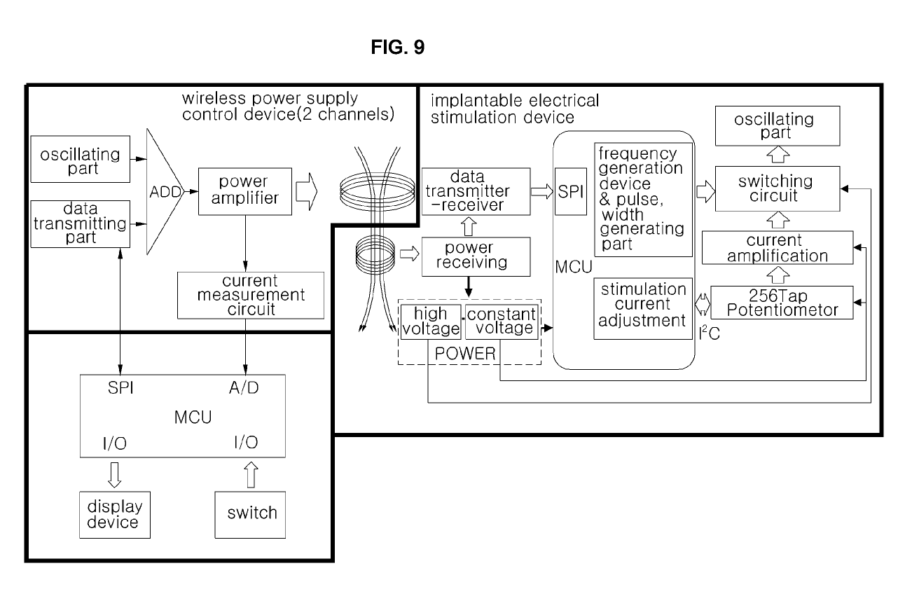

FIG. 1 is a perspective view showing an overall configuration of implantable electrical stimulation device having wireless power supply control function according to the present invention, FIG. 2 is a sectional view showing an electrical stimulation device according to the present invention, which is implanted into cerebral cortex, FIG. 3 is a rear view showing a head set according to the present invention, FIG. 4(a) is a perspective view showing an implantable electrical stimulation device according to the present invention, FIG. 4(b) is a bottom view showing an implantable electrical stimulation device according to the present invention, FIG. 5 is a sectional view showing an implantable electrical stimulation device according to the present invention, FIG. 6 is a sectional view showing a reference electrode a stimulation electrode of an electrical stimulation device according to the present invention, FIG. 7 is a sectional view showing a stimulation electrode of a electric stimulation device according to the present invention, FIG. 8 is a sectional view showing a reference electrode of an electrical stimulation device according to the present invention, and FIG. 9 is a view showing configurations of a wireless power supply control part and an implantable electrical stimulation device according to the present invention.

An implantable cortical electrical stimulation device having wireless power supply control function, as shown FIGS. 2-5, comprises an electrical stimulation element 100 which is arranged on a perforation part of a cranial bone 1 and upper part of which is covered with a scalp so that electrical stimulation is transferred outside dura meter to cerebral cortex 3, a head set 200 which is provided controllably with antenna 210 connecting electrically with the electrical stimulation device 100 outside scalp, and is fixed to a head, and a control element 300 which controls the electrical simulation device 100 through a power wire 211 connected to the antenna 210 of the head set 200.

Here, the electrical stimulation device 100 comprises an electrode bolt 110 which is fastened through the perforation part of a cranial bone (1), a power supply element 120 which is formed on upper part of the electrode bolt 110 and an electrode plate 120 of a plate shape which is formed on lower surface of the electrode bolt 110. Meanwhile, a reference electrode 111 and a stimulation electrode 112 are arranged on both inner lower sides of the electrode bolt 110.

Additionally, the reference electrode 111 and stimulation electrode 112 are same shaped and are made of stainless steel, and further a reference electrode plate 111a and a stimulation electrode plate 112a contacting with cerebral cortex are threaded, respectively. Here, silicon couplings 111b, 112b are inserted in input parts of threaded reference electrode and stimulation electrode plates 111a, 112a, respectively. In addition, an electrical stimulation circuit board 113 surrounded with polyurethane 114 is arranged between the reference electrode and stimulation electrode 111, 112.

Meanwhile, inside of the power supply part 120 is partitioned with polyurethane 114 and a coil 121 is arranged on upper part thereof and a circuit board 122 for controlling stimulation pattern is arranged on the lower part thereof, and the coil 121 is connected electrically to the circuit board 122 for controlling stimulation pattern through power wire 122a. Additionally, the electrical stimulation circuit board 113 is connected electrically to the circuit board 122 for controlling stimulation pattern through power wire 122a. Here, the electrical stimulation circuit board 113 is connected electrically to the reference electrode plate 111a and the stimulation electrode plate 112a through the power wire 122a.

Furthermore, outer part of the electrical stimulation device 100 as configured in an aforementioned way is coated with silicon 130.

FIGS. 6-8 show an electrical stimulation device 100 which is installed in a cranial bone and in which a reference electrode and a stimulation electrode is arranged to be separated. Here, an electrical stimulation device provided with one reference electrode is connected electrically to two stimulation electrodes through ground connection connector. That is, a first electrical stimulation device 150 provided with one reference electrode plate 151, and a second and third electrical stimulation devices 160, 170 which have stimulation electrode plates 161, 171, respectively, and arranged on both sides of the first stimulation device are buried separately into a scalp. Here, the second and third electrical stimulation devices 160, 170 are connected electrically to the first electrical stimulation device 150, respectively, through a ground connection connector 180. Here, configurations of the second and third electrical stimulation devices 160, 170 are similar to that of the electrical stimulation device 100 and thus detailed description thereof is omitted.

Meanwhile, the reference electrode plate 111a and the stimulation plate 112a of the electrical stimulation device 100 are connected symmetrically to the electrode bolt 110, however, the reference electrode plate 151 is connected to the first electrical stimulation device 150, and the stimulation electrode plates 161, 171 are arranged to centers of bolt electrodes of the second and third electrical stimulation devices 161, 171, respectively, and further circuit boards 162, 172 for controlling are inserted into polyurethane and arranged on both sides thereof. Additionally, electrical stimulation circuit boards 163, 173 within urethane are arranged on upper sides of the circuit boards for controlling, and further the electrical stimulation circuit boards 163, 173 is connected to the ground connection connector 180 and connected to a stainless steel connector of the first electrical stimulation device 150.

As shown in FIG. 8, referring to the first electrical stimulation device 150, the reference electrode plate 151 is threaded to bottom of T-shaped stainless steel connector 152.

FIG. 9 shows a configuration of a wireless power supply control part and implantable electrical stimulation device according to the present invention wherein the wireless power supply control part drives the implantable electrical stimulation device, that is, two of the second and third electrical stimulation devices 160, 170. Here, the wireless power supply control part comprises a micro computer controller for controlling a whole system (hereinafter, referred to as “micom”), a power amplifier for supplying power to a antenna, a power measuring member for sensing power, a displaying member for displaying an operation state of the wireless power supply control part and a switch.

Additionally, referring to the implantable electrical stimulation device, mutual induction of external antenna and inner antenna to induce the inner antenna and receives power and power is modulated by ASK modulation method through which digital signal is loaded to analog signal and thus power and data are transmitted simultaneously. A power receiver receives power and supplies to a system and a voltage circuit refers to a circuit supplying power stably to the system, and high voltage part refers to a increasing voltage circuit for producing stimulation voltage. In addition, data transmitter-receiver divides data and transmits them to the micom.

Meanwhile, the micom receiving power and data is a circuit through which stimulation is transmitted to electrode through stimulation voltage control part and frequency and pulse width generation part and stimulation voltage is adjusted minutely to 0.1 voltages using 256 Tap Potentiometer chip. In addition, voltage and current amplifier amplifies the minutely adjusted stimulation voltage such that the stimulation voltage is kept at constant level with respect to a human body. Furthermore, stimulation pattern is transmitted to electrode through a switching circuit to stimulate electrically cerebral cortex.

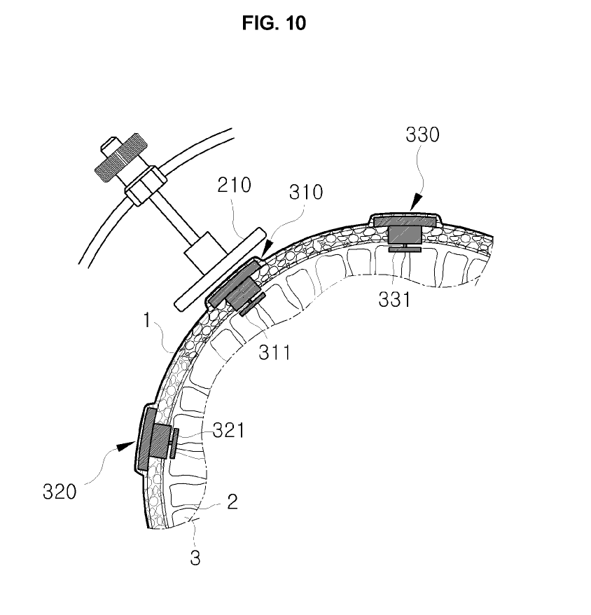

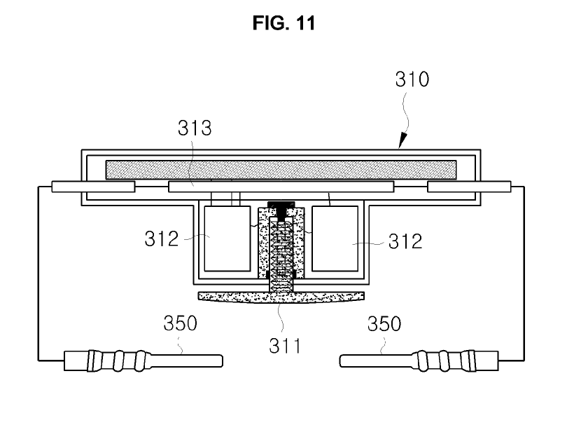

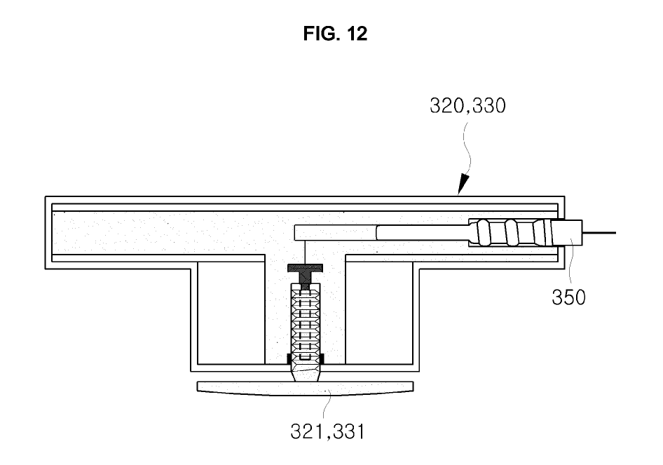

FIG. 10 is a sectional view showing an antenna provided only to a reference electrode of an implantable electrical stimulation device according to another embodiment of the present invention, FIG. 11 is a sectional view showing a reference electrode of an implantable electrical stimulation device according to the present invention, and FIG. 12 is a sectional view showing a stimulation electrode of an implantable electrical stimulation device according to another embodiment of the present invention.

Referring to FIGS. 10-12 show an electrical stimulation device in which a reference electrode and a stimulation electrode of the electrical stimulation device are arranged separately into scalp wherein two of electrical stimulation devices 321, 331 are connected to an electrical stimulation device 310 having one reference electrode plate 311 through stimulation connection connector 350.

That is, the electrical stimulation device 310 having one reference electrode plate 311, and a first and second electrode device 320, 330 having stimulation electrode plates 321, 331, respectively, are buried separately.

Here, stimulation connection connectors 350 which are drawn from both sides of the reference electrode plate 311 are connected to the electrical stimulation device 310, respectively.

Meanwhile, the reference electrode plate 311 is arranged to the electrical stimulation device 310 and stimulation electrode plates 320, 330 are arranged to electrode bolts of the first and second electrode devices 320, 330, respectively. Additionally, a circuit board 312 for controlling is inserted into polyurethane and arranged on both sides of the electrical stimulation device 310 and further a electrical stimulation circuit board 313 within polyurethane is arranged on upper part of the circuit board for controlling and further a pair of stimulation connection connectors 350 are connected to the electrical stimulation circuit board 313 to connected to stainless steel connectors 322, 332 of the first and second electrode devices 320, 330.

Here, two channels, that is, two stimulation connection connectors 350 are connected to the reference electrode plate 311 of the electrical stimulation device 310, and further only one antenna 210 for supplying power wirelessly of the head set 200 is used to the electrical stimulation device. Additionally, a pair of stimulation connection connectors 350 is connected to a pair of stimulation electrode plats 321, 331 to stimulate two channels. Furthermore, the reference electrode plate 311 and the stimulation electrode plates 321, 331 are fastened to electrode through bolt so that their lengths can be adjusted for transferring stimulation properly to cereal cortex.

While the present invention is described referring to the preferred embodiment, the present invention is not limited thereto, and thus various variation and modification can be made without departing from a scope of the present invention.

What is claimed is:

1. An implantable cortical electrical stimulation device having wireless power control function comprising:
an electrical stimulation device (100) which is arranged through a perforation part of cranial bone (1) and upper part of which is covered with scalp and thus electrical stimulation is transferred outside dura meter (2) to cerebral cortex (3);
a head set (200) which is provided controllably with antenna (210) connecting wirelessly with the electrical stimulation device (100) outside scalp and applying power, and is fixed to a head; and
a control element (300) which controls the electrical simulation device (100) through a power wire (211) connected to the antenna (210) of the head set (200).

2. An implantable cortical electrical stimulation device having wireless power control function according to claim 1, wherein the electrical stimulation device (100) comprises an electrode bolt (110) which is fastened through the perforation part of a cranial bone (1), a power supply element (120) of a plate shape which is formed on upper part of the electrode bolt (110) and an electrode plate (120) of a plate shape which is formed on lower surface of the electrode bolt (110) and wherein it comprises:
a reference electrode (111) and a stimulation electrode (112) which are arranged symmetrically on both inner lower sides of the electrode bolt (110) and which are same shaped, and to which a reference electrode plate (111a) and a stimulation electrode plate (112a) contacting with cerebral cortex are threaded, respectively;
silicon couplings (111b), (112b) being inserted in input parts of threaded reference electrode and stimulation electrode plates (111a), (112a), respectively;
an electrical stimulation circuit board (113) surrounded with polyurethane (114) which is arranged between the reference electrode and stimulation electrode (111), (112); and coil (121) which is arranged on upper part of the power supply part (120) inside which is partitioned with polyurethane, a circuit board (122) for controlling which is inserted into the lower part thereof, and power wire (122a) which is connected to the circuit board 122 for controlling.

3. An implantable cortical electrical stimulation device having wireless power control function according to claim 1, wherein outer part of the electrical stimulation device (100) is coated with silicon (130).

4. An implantable cortical electrical stimulation device having wireless power control function comprising:
a first electrical stimulation device (150) which has one reference electrode (151) and which is arranged through a perforation part of cranial bone (1) and upper part of which is covered with scalp and thus electrical stimulation is transferred outside dura meter (2) to cerebral cortex (3);
a second and third electrical stimulation devices (160), (170) which have stimulation electrode plates (161), (171), respectively, and arranged throughly into both cranial bone (1) and upper part of which is covered with scalp and thus electrical stimulation is transferred outside dura meter (2) to cerebral cortex (3);
a head set (200) which is provided controllably with antenna (210) connecting electrically with the second and third electrical stimulation devices (160, 170) outside scalp and applying wirelessly power; and
a control element (300) which controls the second and third electrical simulation devices (160, 170) through a power wire (211) connected to the antenna (210) of the head set (200).

5. An implantable cortical electrical stimulation device having wireless power control function according to claim 4, wherein circuit boards (162),(172) for controlling are inserted into polyurethane and arranged on both sides of the reference electrode plate (151) of the first electrical stimulation device (150) and the stimulation electrode plates (161), (171) of the second and third electrical stimulation devices (160), (170), electrical stimulation circuit boards (163), (173) within urethane are arranged on upper sides of the circuit boards (162), (172) for controlling, and further the electrical stimulation circuit boards (163), (173) are connected to the ground connection connector (180) and connected to a stainless steel connector of the first electrical stimulation device (150).

6. An implantable cortical electrical stimulation device having wireless power control function according to claim 5,
wherein the first electrical stimulation device (150), and the electrode plate (151) of the second and third electrical stimulation devices (160),(170) are threaded to lower end of T-shaped stainless steel connector (152).

7. An implantable cortical electrical stimulation device having wireless power control function comprising:
an electrical stimulation device (310) having one reference electrode plate (311), which is arranged through a perforation part of cranial bone (1) and upper part of which is covered with scalp and thus electrical stimulation is transferred outside dura meter (2) to cerebral cortex (3);
second electrode devices (320), (330) having stimulation electrode plates (161), (171), respectively, which are arranged throughly into both cranial bone (1) and upper part of which is covered with scalp and thus electrical stimulation is transferred outside dura meter (2) to cerebral cortex;
a head set (200) which is provided controllably with antenna (210) connecting wirelessly with the electrical stimulation device (100) outside scalp and applying power, and is fixed to a head; and
a control element (300) which controls the electrical simulation device (310) through a power wire (211) connected to the antenna (210) of the head set (200).

8. An implantable cortical electrical stimulation device having wireless power control function according to claim 7, wherein a pair of stimulation connection connectors 350 which is connected to respective stimulation electrode plates (321), (331) of the first and second electrode devices (320), (330), is connected to both sides of the reference electrode plate 311 of the electrical stimulation device (310).
